(19) United States
(12) Patent Application Publication
(10) Pub. No.: US 2025/0064433 A1
(43) Pub. Date: Feb. 27, 2025

(54) CYTOLOGY DEVICES AND RELATED METHODS

(71) Applicants: BOSTON SCIENTIFIC MEDICAL DEVICE LIMITED, Galway (IE); Boston Scientific Scimed, Inc., Maple Grove, MN (US)

(72) Inventors: Deepak Kumar SHARMA, Muzaffarnagar (IN); Paul SMITH, Smithfield, RI (US); Sharath Kumar G, Kanakapura (IN); Scott E. BRECHBIEL, Burlington, MA (US)

(73) Assignees: BOSTON SCIENTIFIC MEDICAL DEVICE LIMITED, Galway (IE); Boston Scientific Scimed, Inc., Maple Grove, MN (US)

(21) Appl. No.: 18/810,610

(22) Filed: Aug. 21, 2024

Related U.S. Application Data
(60) Provisional application No. 63/578,502, filed on Aug. 24, 2023.

(51) Int. Cl. A61B 10/02

(52) U.S. Cl. CPC A61B 10/02; A61B 2010/0216

(57) ABSTRACT

Systems and methods for a medical device are disclosed. For example, the medical device may include a frame configured to transition from a contracted configuration to an expanded configuration, and a cover disposed on the frame. The cover may include a plurality of projections extending radially outward from an outer surface of the cover.

### CROSS-REFERENCE TO RELATED APPLICATIONS

This application claims the benefit of priority to U.S. Provisional Application No. 63/578,502, filed on Aug. 24, 2023, which is incorporated by reference herein in its entirety.

### TECHNICAL FIELD

Various aspects of this disclosure relate generally to cytology devices and related methods thereof. More specifically, embodiments of the disclosure relate to cytology devices for collecting tissue samples, for example, tissue samples within the common bile duct and the pancreatic duct.

### BACKGROUND

Endoscopic retrograde cholangiopancreatography (ERCP) is a procedure that utilizes endoscopy and/or fluoroscopy to diagnose and/or treat conditions of the biliary and pancreatic ductal systems, such as strictures. During an exemplary ERCP procedure, an endoscope (e.g., a duodenoscope) may be inserted into a subject's mouth and navigated down the esophagus, through the stomach, into the duodenum, and to the papilla, where each of the common bile duct (of the biliary ductal system) and the pancreatic duct enter the duodenum. An operator may cannulate a subject's papilla to access a subject's biliary tree. A contrast agent may then be injected, so that an operator may use fluoroscopy and visualize a stricture within the biliary or pancreatic ductal systems on an x-ray image. The operator may perform a procedure on the biliary tree. For example, the operator may use an instrument to collect a sample of tissue from the stricture to help diagnose malignant stenosis.

### SUMMARY

Each of the aspects disclosed herein may include one or more of the features described in connection with any of the other disclosed aspects.

Aspects of the disclosure relate to a medical device that may include a frame configured to transition from a contracted configuration to an expanded configuration and a cover disposed on the frame. The cover may include a plurality of projections extending radially outward from an outer surface of the cover.

Any of the devices disclosed herein may include any of the following features, in any combination. The medical device may include a control element coupled to the frame. The movement of the control element may be configured to transition the frame from the contracted configuration to the expanded configuration. The control element may be a hypotube configured to receive a guidewire. The frame may include a first, proximal plurality of struts and a second, distal plurality of struts. An inner surface of the cover may be coupled to the second, distal plurality of struts. The distal ends of the second, distal plurality of struts may be coupled to a clevis. The clevis may include a lumen configured to receive a guidewire. The cover may include an opening. The opening may be aligned with the lumen of the clevis. The plurality of projections may include at least a first projection having a first length and at least a second projection having a second length. The first length may be smaller than the second length. The plurality of projections may include a first plurality of projections having the first length and a second plurality of projections having the second length. The first plurality of projections may be distal to the second plurality of projections. The plurality of projections may include at least a first projection having a first stiffness and at least a second projection having a second stiffness. The first stiffness may be greater than the second stiffness. The cover may have a substantially conical shape in the expanded configuration of the frame. The cover may have a substantially cylindrical shape in the contracted configuration of the frame. The plurality of projections may be arranged in a plurality of columns. In the expanded configuration, a circumferential distance between adjacent columns of the plurality of columns may be greater at a proximal end of the cover than at a distal end of the cover. At the proximal end of the cover, the circumferential distance between adjacent columns of the plurality of columns may be greater in the expanded configuration than in the contracted configuration. The cover may be stretchable. The medical device may be configured to receive a guidewire along a central longitudinal axis of the frame.

In another example, a medical device may comprise a handle, a shaft, and a distal end coupled to a distal end of the shaft and configured to expand and contract. The distal end may include a cover. The cover may have a substrate and may include an outer surface with a plurality of projections extending radially outward therefrom.

Any of the devices disclosed herein may include any of the following features, alone or in any combination. The distal end may further include a frame. The cover may be attached to the frame. The substrate may be flexible.

A method of collecting a tissue sample may comprise navigating a medical device to a target site within a body lumen of a subject, moving a control member proximally in order to transition the distal end of the medical device from a contracted configuration to an expanded configuration, and, with the distal end in the expanded configuration, contacting a wall of the body lumen with the plurality of projections to collect the tissue sample. The medical device may include a distal end having a frame and a cover disposed on the frame. The cover may include a plurality of projections extending outward therefrom.

Any exemplary methods disclosed herein may include any of the following steps, alone or in any combination. The method may further comprise moving the control member distally in order to transition the distal end of the medical device from the expanded configuration to the contracted configuration, and, with the distal end in the contracted configuration, contacting a wall of the body lumen with the plurality of projections to collect the tissue sample.

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “distal” refers to a direction away from an operator/toward a treatment site, and the term “proximal” refers to a direction toward an operator. Some of the figures include arrows labeled “distal” and “proximal,” referring to the distal and proximal directions, respectively. The term “exemplary” is used in the sense of “example,” rather than “ideal.” The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 illustrates a portion of an exemplary medical device in an expanded state, according to aspects of this disclosure.

FIGS. 2A-2B illustrate a distal end of the exemplary medical device of FIG. 1 in a first, expanded configuration.

FIG. 3 illustrates the distal end of the exemplary medical device of FIG. 1 in a second, contracted configuration.

FIG. 4 illustrates a frame of the distal end of the medical device of FIG. 1.

FIGS. 5A-5B illustrate a portion of a distal end of the medical device of FIG. 1.

FIG. 6 illustrates an exemplary handle of the medical device of FIG. 1.

FIG. 7 depicts an alternative distal end of a medical device.

FIG. 8 illustrates an alternative frame for use in lieu of the frame of FIG. 4.

FIG. 9 illustrates a related method of using an exemplary medical device, such as the medical devices of FIGS. 1-8.

FIGS. 10-12 illustrate aspects of the exemplary method of FIG. 8.

### DETAILED DESCRIPTION

Embodiments of this disclosure relate to medical devices including structures configured to collect tissue samples from lumen within the body of a subject (for example, within the common bile duct and/or the pancreatic duct) during a medical procedure, for example during an endoscopic retrograde cholangiopancreatography (ERCP) procedure. During an ERCP procedure, a physician or other operator (hereinafter collectively referred to as an “operator”), may utilize both endoscopic and fluoroscopic techniques to diagnose and treat issues arising in the common bile duct and/or in the pancreatic duct. In some instances, these issues may include the presence of strictures, which may need to be analyzed to determine whether they are malignant.

Diagnosis procedures can involve removal of suspected cancer by collection of a sampling of a mucosal tissue layer off the surface of a lumen within the biliary track. For example, an operator may utilize a cytology brush to obtain a sample of cells of the tissue. Present cytology brushes may be off-centered with respect to a guidewire, leading to difficulties in crossing a stricture. Present brushes also may be limited in their abilities to sample irregular surfaces. Poor sampling may lead to, for example, false negative diagnoses.

Although ERCP procedures are referenced herein, it will be appreciated that the disclosed devices and methods may be utilized in the course of other medical procedures (for example, during a variety of medical procedures that involve obtaining biopsy samples of lesions).

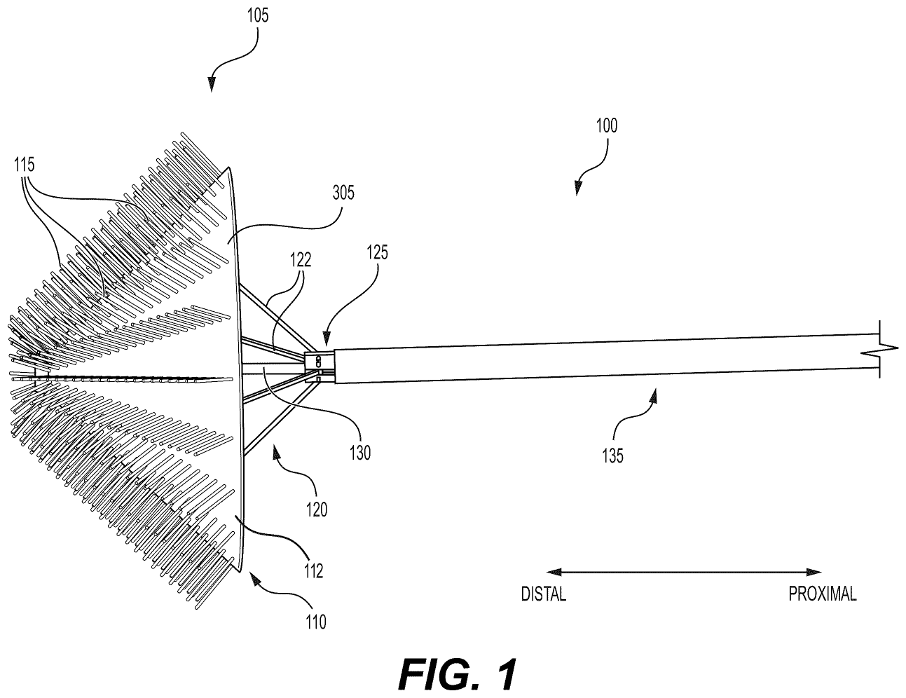

FIGS. 1-7 depict a medical device 100 that may be configured to fit over a guide wire (not shown). Medical device 100 may be similar to a cytology brush. Medical device 100 may have a distal end that collapses, contracts, etc. and/or expands via a frame. In an expanded configuration, distal end of medical device 100 may have a cone-like shape. Medical device 100 may be configured to have a controlled angle of tissue interaction and to deploy to a treatment site, collect a sample of tissue at the treatment site by motion via the controlled angle of tissue interaction, and to return, being removed from the body of a subject such that the sample can be collected and analyzed by a medical professional(s) to produce a test result (e.g., biopsy, etc.).

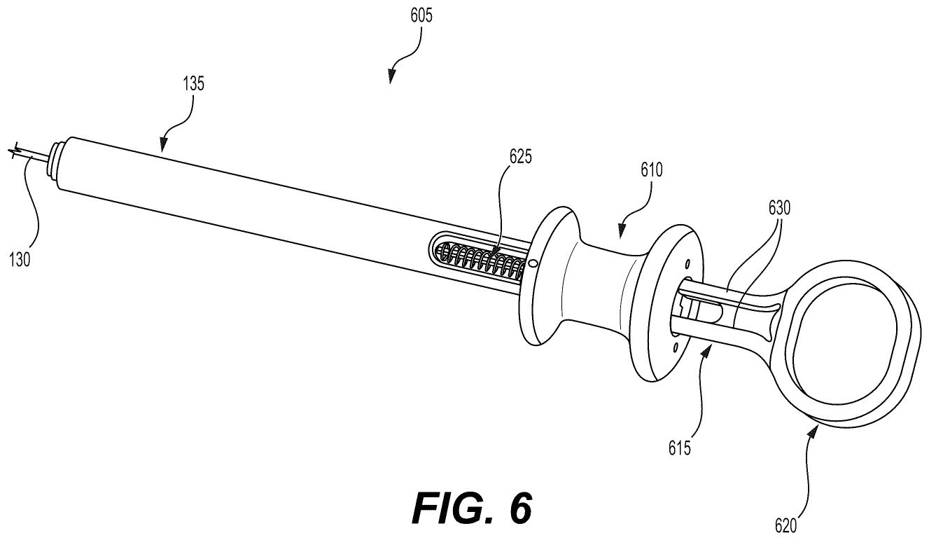

Medical device 100 may include a distal end 105 (including, e.g., a cover 110 and a frame 120), a control member 130, a shaft 135, and/or a control device 605 (described in more detail below with reference to FIG. 6). Cover 110 may be configured to expand and collapse, e.g., in response to proximal or distal movement of control member 130, as described in more detail below. Cover 110 may be comprised of any suitable material, such as at least one polymer or a combination of polymers (e.g., nylon, polytetrafluoroethylene (PTFE), polyether ether ketone (PEEK), etc.).

Shaft 135 may extend from a control device 605 (FIG. 6) to distal end 105. Shaft 135 may have any properties of any shaft of a medical device. Shaft 135 may include, for example, a coil, which may optionally be covered by an outer sheath (e.g., a polymer outer sheath). Shaft 135 may define a lumen (not shown), which may house at least one element of medical device 100, such as a control member 130. Shaft 135 may extend from distal end of control device 605 to a proximal end of a proximal clevis 125 of frame 120, discussed in further detail below. Shaft 135 may be fixedly coupled to proximal clevis 125.

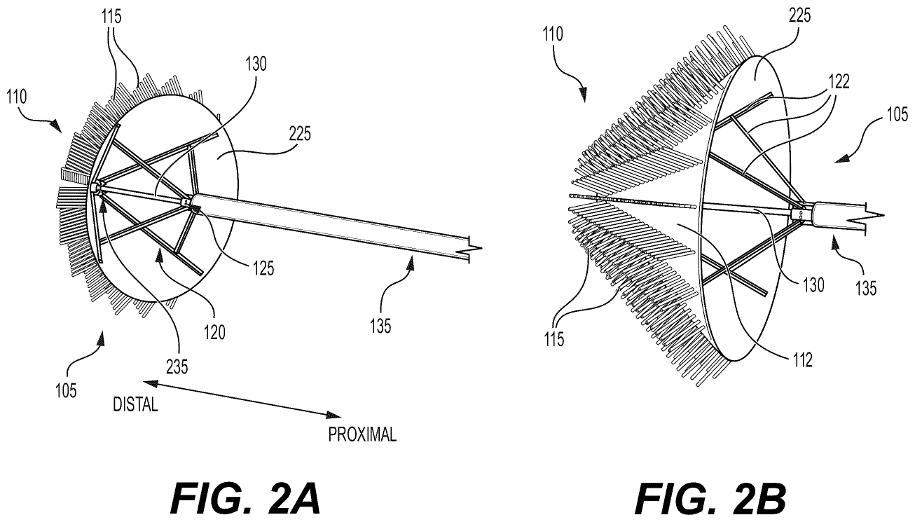

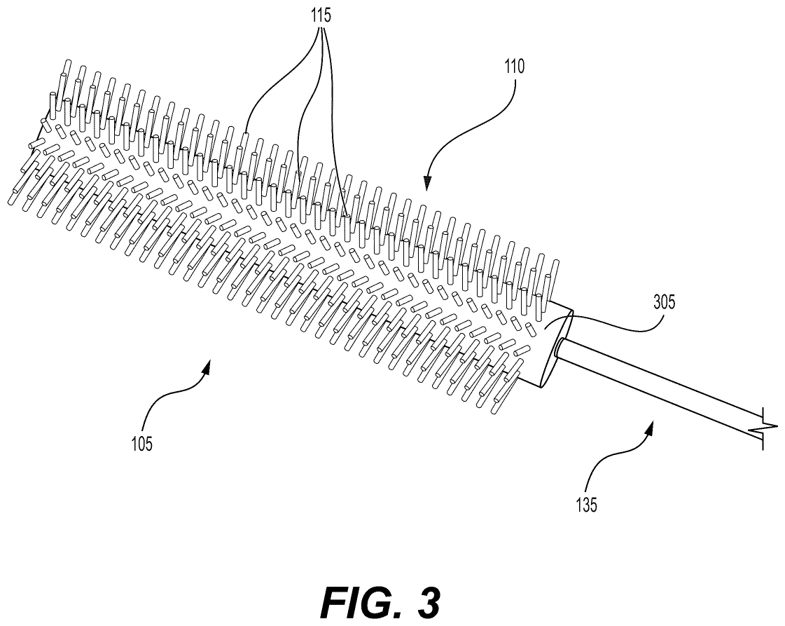

Distal end 105 may include a sheath or cover 110 positioned or disposed over a frame 120. Distal end 105 may be configured to transition between a first, expanded configuration (FIGS. 1A-2B) and a second, contracted, collapsed, etc. configuration (FIG. 3). In some examples, distal end 105 may have an umbrella-like configuration. As shown in FIGS. 1A-2B, when distal end 105 is in the expanded configuration, cover 110 may have an approximately or substantially conical shape. In the expanded configuration, cover 110 may have any suitable dimensions (e.g., any suitable base radius, aperture, height, etc.). For example, a diameter of cover 110 in the contracted configuration may be approximately 1.0-4.0 millimeters, and the diameter of proximal end of cover 110 in the expanded configuration may be approximately 1.0-4.0 millimeters. Cover 110 may be approximately 5.0-20.0 millimeters in length. Substrate 305 of cover 110 may have a thickness of approximately 0.1-0.5 millimeters. Alternatively, cover 110 may have any suitable shape in the expanded configuration (e.g., a truncated cone shape, a pyramid shape, a dome shape, or any other suitable shape).

FIG. 3 depicts distal end 105 in the contracted configuration. As shown in FIG. 3, cover 110 may have an approximately or substantially cylindrical or tubular shape in the contracted configuration. In some examples, cover 110 may be elastic or otherwise stretchable, such that cover 110 is stretched as it transitions from the contracted configuration to the expanded configuration. Cover 110 may have shape-memory properties, such that, when cover 110 is transitioned to the contracted configuration, cover 110 adopts an approximately or substantially cylindrical or tubular shape. Alternatively, cover 110 may not be stretchable, and cover 110 may have a pleated shape in the contracted configuration, similar to a closed umbrella.

Cover 110 may include a substrate 305 with plurality of projections 115 (e.g., bristles) thereon. Substrate 305 may be flexible and may be formed from any suitable material. In some examples, as discussed above, substrate 305 may be elastic or otherwise stretchable. Alternatively, substrate 305 may not be stretchable/elastic. Substrate 305 may form a sheet of material (e.g., a single, continuous sheet of material). Substrate 305 may extend between projections 115 to form a barrier between frame 120 and a space distal of cover 110.

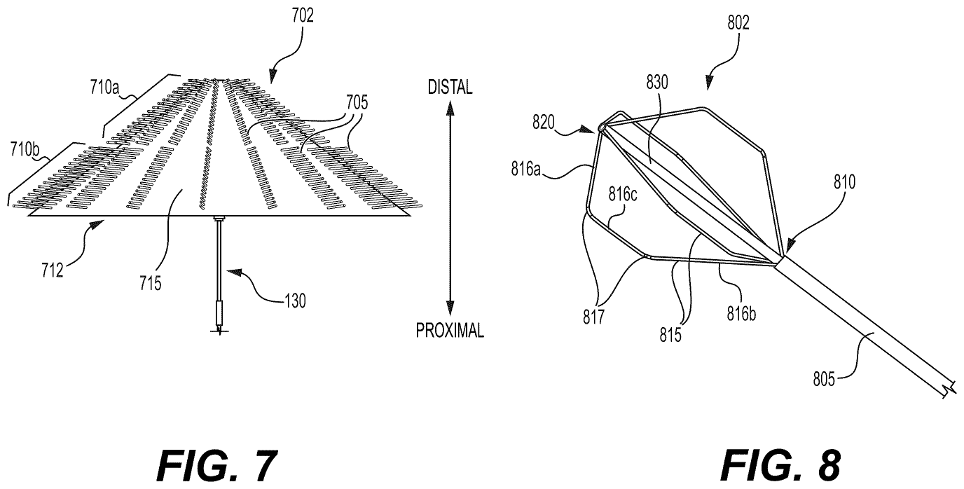

Plurality of projections 115 may have any suitable properties. In some examples, projections 115 may be formed of a same material as substrate 305. In alternatives, projections 115 may be formed from a different material from substrate 305and attached to substrate 305. In some examples, projections 115 may be formed from nylon. In some examples, projections 115 may be uniform, and each of projections 115 may have the same properties. Alternatively, projections 115 may have different properties and may vary in size (length or thickness/diameter), stiffness/durometer, shape, etc., as discussed, for example, with respect to FIG. 7, below. In examples, projections 115 of plurality of projections 115 may increase in stiffness and/or length from a distal end of cover 110 to a proximal end of cover 110, or vice versa. For example, a width/diameter of projections 115, a length of projections 115, and/or a material of projections 115 may vary in order to vary stiffness of projections 115.

As shown in FIGS. 1-3, projections 115 may be arranged in a plurality of columns, extending from the proximal end of cover 110 to distal end of cover 110. As shown in FIG. 3, in the contracted configuration, projections 115 may be approximately evenly spaced on a surface of cover 110, such that a distance between adjacent plurality of projections 115 is approximately uniform, in circumferential and/or longitudinal directions of cover 110. In the expanded configuration (FIGS. 1-2B), projections 115 may have a greater circumferential spacing at the proximal end of cover 110 than at distal end of cover 110. A circumferential distance between circumferentially adjacent plurality of projections 115 may taper downward from the proximal end of cover 110 to distal end of cover 110. In other words, projections 115 may grow gradually closer together along a circumferential direction moving in a distal direction, when cover 110 is in the expanded configuration. Stated differently, the columns of projections 115 may be closer to one another at distal end than at the proximal end. This is because cover 110 may stretch more at the proximal end than at distal end as cover 110 is transitioned from the contracted configuration to the expanded configuration. The arrangement of projections 115 described above is merely exemplary, and projections 115 may have any suitable arrangement. For example, projections 115 may be arranged in a helical pattern or any other suitable arrangement.

Plurality of projections 115 may extend outwardly, e.g., radially outwardly, from an outer surface of cover 110 (hereinafter “outer surface”) 112. Plurality of projections 115 may extend outwardly at any suitable angle, e.g., perpendicular to outer surface 112, angled less than 90 degrees toward the proximal end of cover 110 relative to outer surface 112, angled less than 90 degrees toward distal end of cover 110 relative to outer surface 112, etc., as discussed in further detail below (e.g., see FIG. 7). The angle at which plurality of projections 115 extend outwardly from outer surface 112 may remain constant from when cover 110 is in the expanded state to when cover 110 is in a contracted state, and/or the angle at which plurality of projections 115 extend outwardly from outer surface 112 may vary from when cover 110 is in the expanded state to when cover 110 is in a contracted state. For example, plurality of projections 115 may extend perpendicularly outward from outer surface 112 when cover 110 is in the contracted state, and/or plurality of projections 115 may extend at another angle (e.g., a 45 degree angle or any suitable angle) relative to outer surface 112 when cover 110 is in the expanded state. In some examples, as cover 110 transitions from the expanded configuration to the contracted configuration, as discussed above, each of the plurality of projections 115 may move closer to each other, which may improve retention of the sample on the plurality of projections 115, e.g., as medical device 100 is removed from the body lumen of a patient.

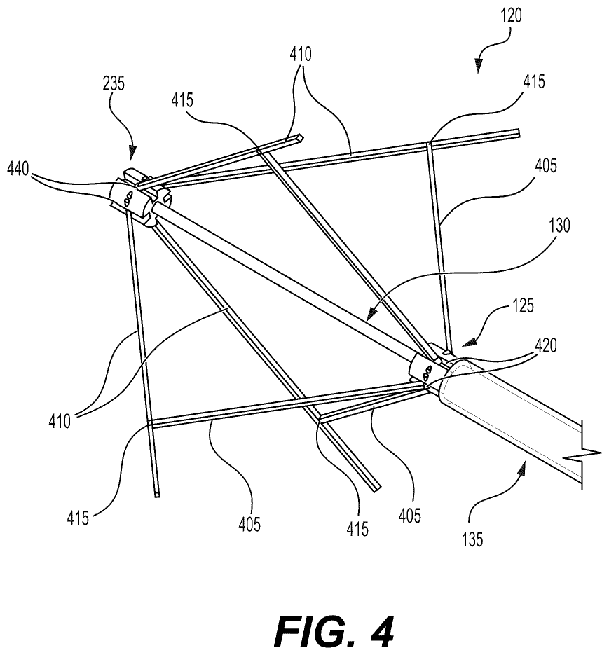

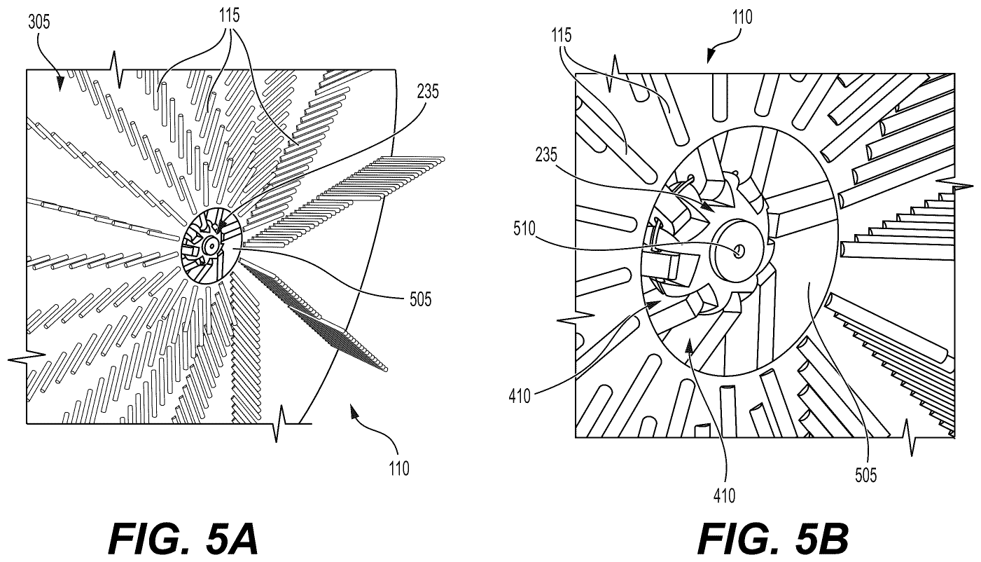

As shown in FIGS. 1-2B and 4, frame 120 may include a plurality of struts 122. In some examples, as depicted in FIG. 4, frame 120 may include a plurality of first struts 405 and a plurality of second struts 410. Plurality of first struts 405 may be proximal to plurality of second struts 410. As shown in FIG. 4, plurality of first struts 405 may include four struts 122, and plurality of second struts 410 may include four struts 122. Alternatively, any suitable number struts may be utilized. For example, FIGS. 5A and 5B depict plurality of second struts 410 as including eight struts 410 (plurality of first struts 405 may include a corresponding number (e.g., eight) of struts 410). FIG. 4 depicts frame 120 in an expanded configuration, as discussed in further detail below. Each strut of plurality of first struts 405 may be connected to a respective strut of plurality of second struts 410 by a plurality of connection points 415 to form a basket shape. Plurality of connection points 415 may include hinges, wheelbases (e.g., wheels within a track of each of plurality of second struts 410 or within a track of each of plurality of first struts 405), etc.

A proximal end of each of first struts 405 may be coupled to proximal clevis 125 via a plurality of hinges 420. Plurality of hinges 420 of proximal clevis 125 may be configured to allow hinging movement of plurality of first struts 405 with respect to proximal clevis 125. A distal end of each of second struts 410 may be coupled to distal clevis 235 via a plurality of hinges 440. As shown particularly in FIGS. 4, 5A, and 5B, hinges 440 of distal clevis 235 may be configured to enable hinging movement of plurality of second struts 410 with respect to distal clevis 235.

A distal end of shaft 135 may be coupled to a proximal end of proximal clevis 125. Control member 130 may extend from a distal end of shaft 135, through a lumen of proximal clevis 125, through frame 120 along a longitudinal axis of distal end 105, to distal clevis 235. Control member 130 may be movable with respect to proximal clevis 125. Control member 130 may be fixedly coupled to distal clevis 235 (e.g., to a proximal end of distal clevis 235). Control member 130 may be composed of any suitable material, e.g., at least one polymer, a combination of polymers, metal, etc. In some examples, control member 130 may be a hypotube. Alternatively, control member 130 may include a wire, cable, rigid bar, etc.

Proximal movement of control member 130 may cause distal clevis 235 to move proximally, toward proximal clevis 125. As distal clevis 235 moves toward proximal clevis 125, proximal ends of second struts 410 and distal ends of first struts 405 (joined together at connection points 415) may move radially outward. Such movement may cause frame 120 to move from a contracted state to an expanded state. In the expanded state, hinges 420 may be spaced apart from one another, such that frame 120 has a larger width/diameter at a location of hinges 420 than at a proximal end of frame 120 (near proximal clevis 125) and distal end of frame 120 (near distal clevis 235). In the contracted state, connection points 415 may be closer to one another (e.g., adjacent to one another), such that frame 120 has an approximately uniform width/diameter near proximal clevis 125, at hinges 420, and near distal clevis 235.

Distal movement of control member 130 may cause distal clevis 235 to move distally, away from proximal clevis 125. As distal clevis 235 moves away from proximal clevis 125, proximal ends of second struts 410 and distal ends of first struts 405 (joined together at connection points 415) may move radially inward, closer to one another. Such movement may cause frame 120 to move from the expanded state to the contracted state.

Struts 122 may interact with an inner surface of cover 110 (hereinafter “inner surface”) 225 (see FIGS. 2A and 2B). In some embodiments, cover 110 may be affixed to struts 122 of frame 120. For example, inner surface 225 may be coupled (e.g., fixedly coupled, via adhesive or any other suitable mechanism, such as loops of thread or metal) to each of plurality of first struts 405. When frame 120 is in an expanded state (FIGS. 1-2B, 4), cover 110 may be in an expanded state, as depicted in FIGS. 1-2B. When frame 120 is in a contracted state, cover 110 may be in a contracted state, as depicted in FIG. 3.

Because, in the expanded configuration, proximal ends of plurality of first struts 405 may be further from one another than distal ends of plurality of first struts 405, as discussed above, projections 115 may have a greater circumferential spacing from one another at a proximal end of cover 110 as compared with a distal end of cover 110. In alternative embodiments, frame 120 and/or cover 110 may have alternative shapes. For example, frame 120 may be any structure that expands/contracts, such as a malecot, a basket, etc. One such alternative frame is discussed below, with respect to FIG. 8.

As discussed above, control member 130 may be a hypotube. Control member 130 may define a lumen extending therethrough. In some examples, the lumen may extend from a proximal end of control member 130 to a distal end of control member 130. In alternatives, the lumen may extend to an opening that is distal to a proximal end of control member 130. In some examples, distal clevis 235 may include a lumen 510 extending axially through distal clevis 235. Lumen 510 may have openings on proximal and distal sides of distal clevis 235. In aspects, control member 130 may be affixed to an edge or rim of lumen 510, such that the lumen of control member 130 is in fluid communication with lumen 510. In alternatives, control member 130 may extend through lumen 510 and may be coupled to an inner surface of lumen 510. Cover 110 may also have an opening 505 at or near a center of cover 110. In examples, opening 505 may be aligned with lumen 510, such that lumen 510 is exposed to an area of a subject's body lumen that is distal of medical device 100.

Medical device 100 may be passed over a guidewire. For example, a proximal end of a guidewire may be inserted into or received by the distal opening of lumen 510, and into the lumen of control member 130. Thus, a guidewire may extend through the hypotube lumen of control member 130 and lumen 510 (e.g., a guidewire lumen). The guidewire (not shown) may extend along a central longitudinal axis of medical device 100, through a center of frame 120 and cover 110, through opening 505. Because the guidewire extends along the central longitudinal axis of medical device 100, medical device 100 may have enhanced steering as compared with conventional cytology brushes. For example, it may be easier for an operator to extend medical device 100 into and through strictures.

The expansion and contraction of cover 110 and/or frame 120 may be actuated by a control device 605, as depicted by FIG. 6 Control device 605 may be a handle and may be coupled to a proximal end of shaft 135. Control device 605 may have features of any handle known in the art for controlling medical devices. Control device 605 may include a spool 610, a control device shaft 615, and a thumb loop 620. Control member 130 may be coupled to and extend distally from spool 610. In some examples, as shown in FIG. 6, a coil 625 (e.g., a spring) may extend about control member 130.

An operator may move spool 610 proximally and/or distally along control device shaft 615, toward and away from thumb loop 620. For example, control device shaft 615 may include a track 630 (e.g., a longitudinal opening). Proximal and distal movement of spool 610 may cause proximal and distal movement of control member 130, respectively. As discussed above, proximal movement of control member 130 (via proximal movement of spool 610) may transition distal end 105 from the contracted configuration (FIG. 3) to the expanded configuration (FIGS. 1-2B). Distal movement of control member 130 (via distal movement of spool 610) may transition distal end 105 from the expanded configuration to the contracted configuration.

In some examples, coil 625 may bias medical device 100 to the contracted configuration (a configuration in which spool 610 is located relatively distally, e.g., at a distal end of track 630. When an operator moves spool 610 proximally, coil 625 may be compressed or extended as compared to its natural length. When the operator releases spool 610, coil 625 may return to its natural length, causing control member 130 to move distally, and thereby causing cover 110 and/or frame 120 to move from the expanded state to the contracted state. An arrangement of control device 605 is merely exemplary, and any suitable handle may be utilized.

FIG. 7 depicts an alternative cover 702. Cover 702 may be used in place of cover 110 with medical device 100. Except as described below, cover 702 may have any of the features of cover 110, e.g., the composition and/or shape of the substrate. Cover 702 may include a plurality of projections 705, which may be arranged in a plurality of columns, a helical pattern, or any other suitable arrangement, as discussed in further detail above. Projections 705 may have varying lengths, compositions, angles, etc. For example, as depicted in FIG. 7, a first section of plurality of projections 705 (hereinafter “first section”) 710a may have a different length (e.g., may be smaller or larger) than a second section of plurality of projections 705 (hereinafter “second section”) 710b. For example, as shown in FIG. 7, projections 705 of first section 710a may have a shorter length than projections of second section 710b. As shown in FIG. 7, projections 705 of section 710a may have a first uniform length, and projections 705 of section 710b may have a second uniform length. Alternatively, lengths of projections 705 may be variable within first section 710a and/or second section 710a. For example, projections 705 (e.g., in a helical or columnar configuration) may gradually increase in size from distal end of cover 702 to the proximal end of cover 702, or vice versa.

Plurality of projections 705 may extend outwardly, e.g., radially outwardly, from an outer surface of cover 702 (hereinafter “outer surface”) 715. As discussed above in relation to plurality of projections 115, plurality of projections 705 may extend outwardly at any suitable angle, e.g., perpendicular to outer surface 715, angled less than 90 degrees toward the proximal end of cover 702 relative to outer surface 715, angled less than 90 degrees toward distal end of cover 702 relative to outer surface 715, etc. For example, first section 710a may be perpendicular to outer surface 715 and second section 710b may be angled toward the proximal end of cover 702 at an angle less than 90 degrees relative to outer surface 715.

The angle at which plurality of projections 705 extend outwardly from outer surface 715 may vary from when cover 702 is in the expanded state to when cover 702 is in a contracted state. For example, when cover 110 is in the contracted state, first section 710a may extend perpendicularly outward from outer surface 112 and second section 710b may be approximately parallel with outer surface 715. In another example, first section 710a and second section 710b may extend at other angles (e.g., a 45 degree angle or any suitable angle) relative to outer surface 715 when cover 702 is in the expanded state and/or the contracted state.

Plurality of projections 705 may have any suitable properties. In some examples, projections 705 of first section 710a may be formed of a first material and projections 705 of second section 710b may be formed of a second material. For example, projections 705 of first section 710a may be formed of a same material as substrate 712 and projections second section 710b may be formed of a different material from substrate 712 (e.g., nylon) and attached to substrate 712 (or vice versa). In some examples, plurality of projections 705 may have different properties and may vary in size (length or thickness/diameter), stiffness/durometer, shape, etc. In examples, plurality of projections 705 may increase in stiffness and/or length from a distal end of cover 702 to a proximal end of cover 702, or vice versa. For example, first section 710a may have a greater stiffness than second section 710b, or vice versa.

FIG. 8 depicts an alternative frame 802. Frame 802 may be used in place of frame 120 with medical device 100 and may, in some examples, be combined with cover 702 of FIG. 7. Except as described below, frame 802 may have any of the features of frame 120. As shown in FIG. 8, frame 802 may include a plurality of struts 815, an outer tubular member 805, and an inner tubular member 830. Inner tubular member 830 and/or outer tubular member 805 may have any of the properties of control member 130. Inner tubular member 830 may be movably received within a lumen of outer tubular member 805.

Although frame 802 includes four struts 815 in FIG. 8, it will be appreciated that frame 802 may include any alternative number of struts. Struts 815 may be a plurality of wires comprised of a shape memory material, for example nitinol, but any suitable material may be used. Struts 815 may be biased to an expanded configuration, shown in FIG. 8. In some examples, in the biased, expanded state of struts 815, struts 815 may be wire or may include a plurality of flexion points 817. Flexion points 817 may form living hinges. Alternatively, struts 815 may have any suitable shape in the expanded configuration. As shown in FIG. 8, struts 815 may have a first segment 816a, a second segment 816b, and a third segment 816c. First segment 816a may be a distal segment, second segment 816b may be a proximal segment, and third segment 816c may extend between first segment 816a and second segment 816b. Each of segments 816a, b, c may be straight, as shown in FIG. 8. Alternatively, one or more of segments 816a, b, c may be curved. Struts 815 may have fewer (e.g., two) or more (e.g., four or more) than three segments.

A proximal end of each of struts 815 may be fixedly coupled to a distal end of outer tubular member 805 at a first fixation location 810. A distal end of each of struts 815 may be fixedly coupled to a distal fixation location 820. Inner tubular member 830 may be composed of any suitable material, e.g., at least one polymer, a combination of polymers, metal, etc. In some examples, inner tubular member 830 may be a hypotube. A lumen of inner tubular member 830 may be sized and shaped so as to receive a guidewire or other accessory device, as described above with respect to control member 130. Alternatively, inner tubular member 830 may include a wire, cable, rigid bar, etc. Outer tubular member 805 may similarly be composed of any suitable materials, including any of the materials escribed above for inner tubular member 830. A lumen of outer tubular member 805 may be sized and shaped so as to movably receive inner tubular member 830.

Proximal movement of outer tubular member 805 may cause proximal ends of struts 815 to move proximally, thereby collapsing struts 815 into a contracted configuration (not shown). In the contracted state, struts 815 may be relatively straightened, and flexion points 817 may be close to one another (e.g., adjacent to one another), such that frame 802 may have an approximately uniform width/diameter near first fixation location 810, at plurality of flexion points 817, and near second fixation location 820.

Distal movement of outer tubular member 805 may cause proximal ends of struts 815 to move distally, thereby causing struts 815 to move radially outward, e.g., at plurality of flexion points 817. Such movement may cause frame 802 to move from the contracted state to the expanded state, shown in FIG. 8. In the expanded state, struts 815, including plurality of flexion points 817, may be spaced apart from one another, such that frame 802 has a larger width/diameter at third segments 816c than at a proximal end of frame 802 (near first fixation location 810) and distal end of frame 802 (near second fixation location 820). In the expanded state of FIG. 8, first and second segments 816a, 816b may be angled relative to third segment 816c.

Plurality of struts 815 may interact with inner surface 225 or an inner surface of cover 702. In some embodiments, cover 110 or cover 702 may be affixed to plurality of struts 815 of frame 802. For example, inner surface 225 or the inner surface of cover 702 may be coupled (e.g., fixedly coupled, via adhesive or any other suitable mechanism, such as loops of thread or metal) to each of struts 815. When frame 802 is in an expanded state, cover 110 or cover 702 may be in an expanded state, similar to that depicted in FIGS. 1-2B and FIG. 7. When frame 802 is in a contracted state, cover 110 or cover 702 may be in a contracted state, similar to that depicted in FIG. 3.

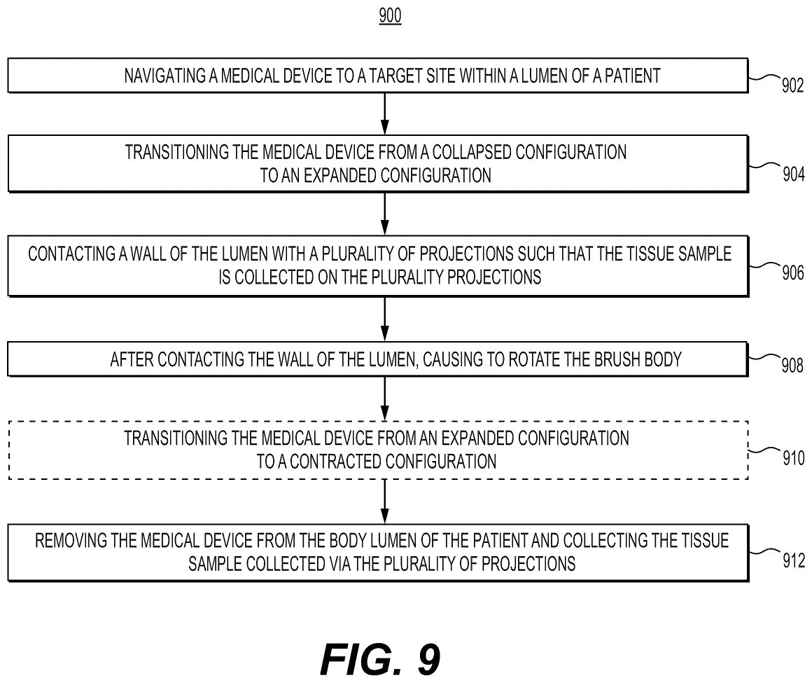

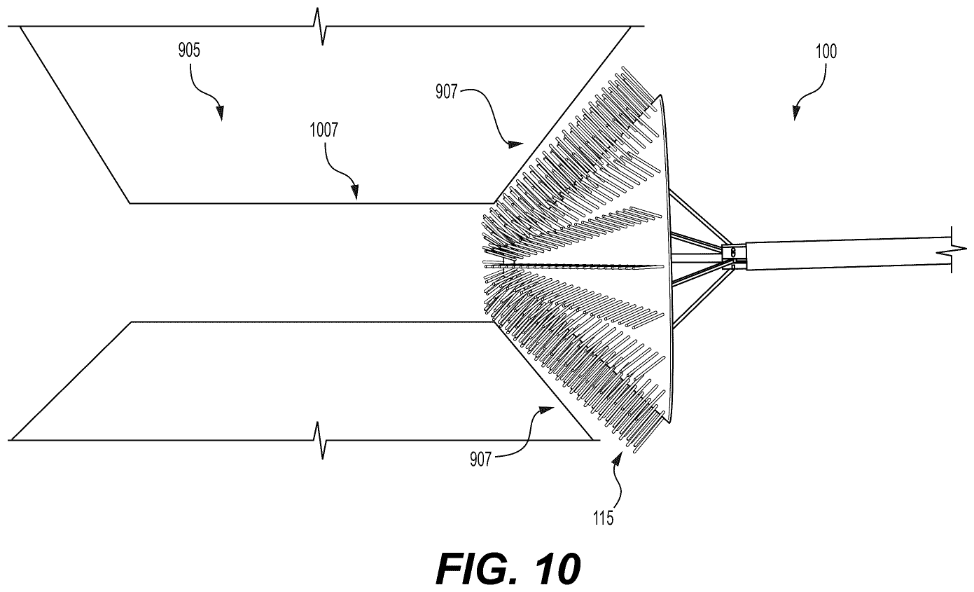

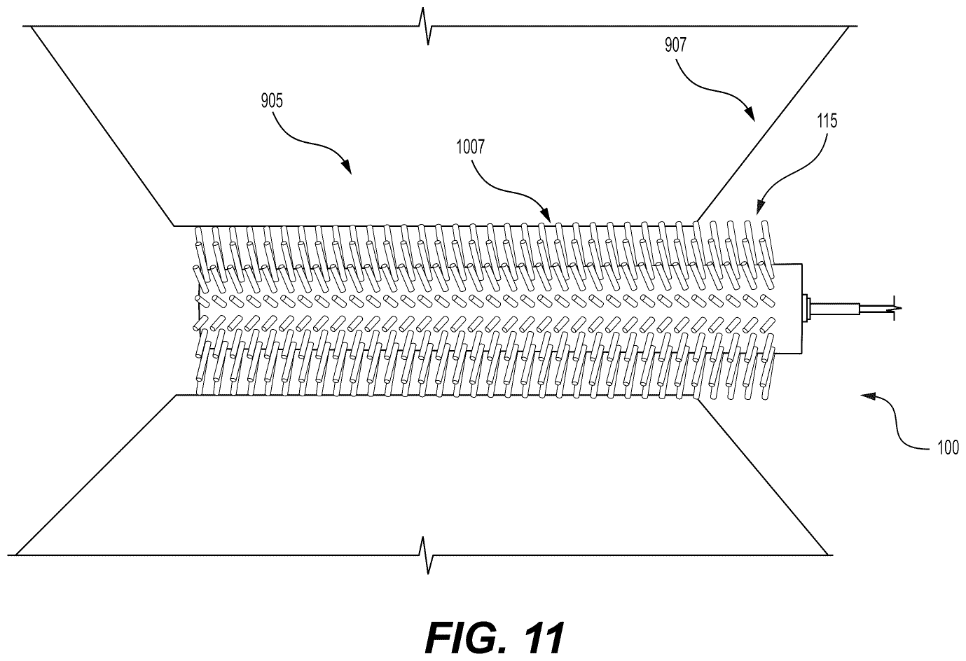

FIG. 9 illustrates a related method 900 of using an exemplary medical device, such as medical device 100 (including use with cover 110, frame 120, cover 702 of FIG. 7 and/or frame 802 of FIG. 8). FIGS. 10 and 11 depict aspects of method 900. At step 902, the medical device may be navigated to a target site within a body lumen of a subject. In some examples, a duodenoscope or other type of scope may be utilized. A guidewire may have previously been positioned so as to provide access to the target site. Step 902 may include inserting the guidewire within lumen 510 (or a corresponding lumen of frame 802 of FIG. 8), and extending the guidewire through the lumen of control member 130. The medical device may be advanced over the guidewire to a target site. Thus, the medical device may have a controlled angle of tissue attack. As In some examples, medical device 100 may be in the contracted state while being moved to the target site, e.g., to avoid plurality of projections interacting with other sites within the lumen and potentially collecting an undesired tissue sample. In some examples, step 902 may include navigating the medical device to a proximal wall 907 of a stricture 905, as shown in FIG. 10.

At step 904, medical device 100 may be transitioned from a contracted configuration to an expanded configuration. As discussed herein, an operator may move spool 610 proximally, causing control member 130 to move proximally. As control member 130 moves proximally, distal clevis 235 may be caused to move proximally, e.g., toward proximal clevis 125, thereby causing frame 120 to move outward laterally relative to control member 130 and/or a central longitudinal axis of distal end 105, as described above. As frame 120, e.g., plurality of connection points 415, moves laterally outward, cover 110 may be caused to move from a contracted state to and expanded state.

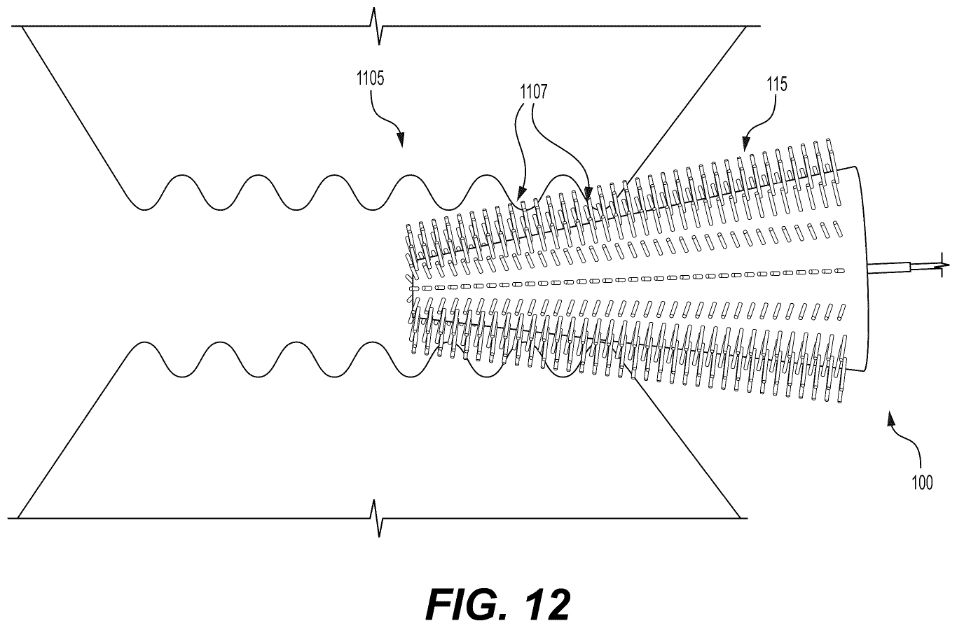

In some examples, cover 110 may be in a partial expanded state. For example, cover 110 may be caused to expand 10%, 25%, 50%, 75%, etc. In other words, cover 110 may expand part way between the contracted state and the expanded state, e.g., dependent upon the amount of proximal movement of spool 610. An example of cover 110 in a partially expanded state is depicted in FIG. 12, which is discussed in more detail below.

At step 906, a wall of the lumen may be contacted with plurality of projections 115 such that the tissue sample is collected on plurality of projections 115. Step 906 may include contacting proximal wall 907, as shown in FIG. 10. Additionally or alternatively (e.g., subsequently to contacting proximal wall 907), step 906 may include advancing distal end 105 to be within stricture 905 and contacting a wall within stricture 905 (e.g., an internal portion of a stricture (hereinafter “internal portion”) 1007).

At step 908, after contacting the wall of the lumen, distal end 105 (e.g., including cover 110 and plurality of projections 115) may be caused to be rotated. For example, as depicted in FIG. 10, plurality of projections 115 may be moved against, e.g., rotated against, stricture 905 (e.g., a biliary stricture), proximal wall 907, etc.

Optionally, at step 910, medical device 100 may be transitioned from an expanded configuration to a contracted configuration. Upon completion of the transition, any of steps 904-908 may be repeated. For example, as depicted in FIG. 10, medical device 100 may be transitioned to the contracted state to make contact with internal portion 1007.

While internal portion 1007 is depicted as relatively uniform, in some instances, such as depicted in FIG. 11, a stricture, e.g., stricture 1105, may include an irregularly shaped internal portion (hereinafter “irregular internal portion”) 1107. In some examples, medical device 100 may be moved iteratively between the contracted state, the expanded state, and any partial degree of contraction or expansion therebetween in order for the operator to contact surfaces having varying shapes, advantageously improving tissue sample quality and reducing false negatives from the collected tissue sample.

At step 912, medical device 100 may be removed from the body lumen of the subject and the tissue sample collected via plurality of projections 115 may be collected. In some examples, cover 110 may be removed from plurality of projections 115 by disconnecting the one or more detachable connection points. In some examples, distal end 105 may be removed from medical device 100 by severing a portion between cover 110 and spool 610. For example, distal end 105 may be removed from shaft 135 (e.g., at proximal clevis 125).

While principles of this disclosure are described herein with reference to illustrative examples for particular applications, it should be understood that the disclosure is not limited thereto. For example, the disclosure refers to ERCP as an exemplary procedure, and the bile and pancreatic ducts as typical lumens for the systems and methods of the disclosure. The systems, devices, and methods of the present disclosure, however, may be used in any suitable medical procedure in any lumen or cavity within the body, for example, to remove any unwanted material from the body. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A medical device, comprising:
a frame configured to transition from a contracted configuration to an expanded configuration; and
a cover disposed on the frame, wherein the cover includes a plurality of projections extending radially outward from an outer surface of the cover.

2. The medical device of claim 1, further comprising a control element coupled to the frame, wherein movement of the control element is configured to transition the frame from the contracted configuration to the expanded configuration, and wherein the control element is a hypotube configured to receive a guidewire.

3. The medical device of claim 1, wherein the frame includes a first, proximal plurality of struts and a second, distal plurality of struts, and wherein an inner surface of the cover is coupled to the second, distal plurality of struts.

4. The medical device of claim 3, wherein distal ends of the second, distal plurality of struts are coupled to a clevis, and wherein the clevis includes a lumen configured to receive a guidewire.

5. The medical device of claim 4, wherein the cover includes an opening, and wherein the opening is aligned with the lumen of the clevis.

6. The medical device of claim 1, wherein the plurality of projections include at least a first projection having a first length and at least a second projection having a second length, wherein the first length is smaller than the second length.

7. The medical device of claim 6, wherein the plurality of projections include a first plurality of projections having the first length and a second plurality of projections having the second length, and wherein the first plurality of projections is distal to the second plurality of projections.

8. The medical device of claim 1, wherein the plurality of projections include at least a first projection having a first stiffness and at least a second projection having a second stiffness, wherein the first stiffness is greater than the second stiffness.

9. The medical device of claim 1, wherein the cover has a substantially conical shape in the expanded configuration of the frame.

10. The medical device of claim 9, wherein the cover has a substantially cylindrical shape in the contracted configuration of the frame.

11. The medical device of claim 9, wherein the plurality of projections are arranged in a plurality of columns.

12. The medical device of claim 11, wherein, in the expanded configuration, a circumferential distance between adjacent columns of the plurality of columns is greater at a proximal end of the cover than at a distal end of the cover.

13. The medical device of claim 12, wherein, at the proximal end of the cover, the circumferential distance between adjacent columns of the plurality of columns is greater in the expanded configuration than in the contracted configuration.

14. The medical device of claim 1, wherein the cover is stretchable.

15. The medical device of claim 1, wherein the medical device is configured to receive a guidewire along a central longitudinal axis of the frame.

16. A medical device, comprising:
a handle;
a shaft; and
a distal end coupled to a distal end of the shaft and configured to expand and contract, the distal end including a cover having:
a substrate; and
a cover including an outer surface with a plurality of projections extending radially outward therefrom.

17. The medical device of claim 16, wherein the distal end further includes a frame, and wherein the cover is attached to the frame.

18. The medical device of claim 16, wherein the substrate is flexible.

19. A method of collecting a tissue sample, comprising:
navigating a medical device to a target site within a body lumen of a subject, wherein the medical device includes a distal end having a frame and a cover disposed on the frame, wherein the cover includes a plurality of projections extending outward therefrom;
moving a control member proximally in order to transition the distal end of the medical device from a contracted configuration to an expanded configuration; and
with the distal end in the expanded configuration, contacting a wall of the body lumen with the plurality of projections to collect the tissue sample.

20. The method of claim 19, further comprising:
moving the control member distally in order to transition the distal end of the medical device from the expanded configuration to the contracted configuration; and
with the distal end in the contracted configuration, contacting a wall of the body lumen with the plurality of projections to collect the tissue sample.
